(19) United States
(12) Patent Application Publication
(10) Pub. No.: US 2024/0261524 A1
(43) Pub. Date: Aug. 8, 2024

(54) VENTILATION SYSTEM FOR ARTIFICIAL VENTILATION WITH A VOLUME FLOW DISPLAY ELEMENT

(71) Applicant: Drägerwerk AG & Co. KGaA, Lübeck (DE)

(72) Inventors: Robert SCHMID, Lübeck (DE); Guido WAGENER, Lübeck (DE)

(21) Appl. No.: 18/432,207

(22) Filed: Feb. 5, 2024

(30) Foreign Application Priority Data
Feb. 7, 2023 (DE) 10 2023 102 932.0

(51) Int. Cl. A61M 16/00

(52) U.S. Cl. CPC A61M 16/024; A61M 16/0003; A61M 16/0066

(57) ABSTRACT

A ventilation system for artificial ventilation of a patient (Pt) includes an inspiratory fluid guide unit (30) that connects a ventilator (1) to a patient-side coupling unit (9). A control unit (4) determines a measure for a net inspiratory volume flow which the ventilator (1) generates and which flows through the inspiratory fluid guide unit (30). Depending on the net inspiratory volume flow, the control unit (4) controls an inspiratory display element (40) on the inspiratory fluid guide unit (30). The actuated inspiratory display element (40) displays two indicators in a visually perceptible manner, namely an indicator for the determined net inspiratory volume flow and an indicator for the direction of flow through the inspiratory fluid guide unit (30).

### CROSS REFERENCE TO RELATED APPLICATIONS

This application claims the benefit of priority under 35 U.S.C. § 119 of German Application 10 2023 102 932.0, filed Feb. 7, 2023, the entire contents of which are incorporated herein by reference.

### TECHNICAL FIELD

The invention relates to a ventilation system which is capable of artificially ventilating a patient and which comprises at least one display element (indicator element) which indicates the direction of flow of a gas.

### BACKGROUND

A ventilation system that is capable of artificially ventilating a patient usually comprises a ventilator, a patient-side coupling unit and an inspiratory fluid guide unit. The patient-side coupling unit is arranged in and/or on the patient's body and comprises, for example, a breathing mask and/or a tube and/or a catheter. The inspiratory fluid guide unit is capable of guiding a fluid from the ventilator to the patient-side coupling unit and comprises, for example, a tube and optionally also a pipe. The ventilator performs a sequence of ventilatory strokes, wherein the ventilator ejects a quantity of a gas mixture in each ventilatory stroke and the ejected quantity is conducted from the ventilator through the inspiratory fluid guide unit to the patient-side coupling unit. The gas mixture comprises oxygen. The ventilation system according to the invention also uses this principle.

In one form, the gas mixture additionally comprises at least one anesthetic agent. Therefore, the patient is sedated or anesthetized. In particular in this case, the ventilation system generally establishes a ventilation circuit. The ventilator and optionally the patient's own respiratory activity maintain this ventilation circuit. The gas mixture exhaled by the patient usually contains anesthetic and is fed back to the ventilator by the patient-side coupling unit through an expiratory fluid guide unit. Thanks to the ventilation circuit, this anesthetic does not escape into the environment. In one application, the ventilation system according to the invention also uses this principle.

It is desirable that an anesthesiologist or other user can quickly determine whether the patient is actually being artificial ventilated as desired or whether an error or malfunction has occurred.

### SUMMARY

It is an object of the invention to provide a ventilation system which in many cases makes it easier for a user to monitor the ventilation system.

The object is attained by a ventilation system with features disclosed herein. Advantageous embodiments of the ventilation system according to the invention are disclosed herein.

The ventilation system according to the invention comprises a ventilator. The ventilator is capable of ejecting (expelling, supplying) a gas mixture. This gas mixture comprises oxygen. In one embodiment, the gas mixture is pure oxygen. In a preferred embodiment, the gas mixture comprises at least one further gas component, for example breathing air and/or at least one anesthetic agent. An anesthesia device is a possible embodiment of a ventilator as defined in the claims. Preferably, the ventilator performs a sequence of ventilation strokes and ejects a quantity of the gas mixture in each ventilation stroke.

The ventilation system also includes a patient-side coupling unit. The patient-side coupling unit is at least temporarily connected to the patient or can be connected to the patient. A breathing mask, a tube, and a catheter are configurations and/or possible components of the patient-side coupling unit.

An inspiratory fluid guide unit of the ventilation system is configured to guide a gas mixture, which the ventilator has ejected, to the patient-side coupling unit. A “fluid guide unit” is a component that is configured to guide a fluid along a trajectory specified by the configuration and arrangement of the component and ideally prevents the fluid from leaving this trajectory. A hose and a tube are two examples of a fluid guide unit.

The ventilation system also comprises an inspiratory display element (an inspiratory indicator element). A “display element” is understood to be a controllable component that is capable of outputting information about the ventilation system in at least one way that can visually be perceived by a person. In a first alternative, this inspiratory display element is attached to the inspiratory fluid guide unit. In a second alternative, the inspiratory display element is attached to another fluid guide unit, said other fluid guide unit being in fluid connection with the ventilator and with the inspiratory fluid guide unit. A “fluid connection” is established between two components if a fluid can at least temporarily flow from one component to the other component.

A signal-processing control unit of the ventilation system is configured to determine a measure of the net inspiratory volume flow. A “volume flow” through a fluid guide unit is the volume per unit of time that flows through this fluid guide unit. As a rule, the volume flow varies over time. Of course, it is possible that at least temporarily no fluid flows through the fluid guide unit and the volume flow is therefore zero or at least below a specified lower threshold.

The “net inspiratory volume flow” is understood to be the volume flow through the inspiratory fluid guide unit that the ventilator generates and ejects, preferably with its ventilation strokes. The total volume flow through the inspiratory fluid guide unit can be a superposition of the net inspiratory volume flow and at least one further volume flow. A further volume flow can be generated in particular by establishing a ventilation circuit such that an expiratory gas mixture flowing from the patient-side coupling unit and through the ventilator back into the inspiratory fluid guide unit. A fluid can also flow through the inspiratory fluid guide unit, which fluid is used to clean a fluid guide unit or to measure the concentration of a gas component in a gas mixture. Furthermore, a leak in the inspiratory fluid guide unit can lead to a volume flow that does not reach the patient-side coupling unit. It is also possible that the total volume flow through the inspiratory fluid guide unit is equal to the net inspiratory volume flow.

The control unit is configured to control the inspiratory display element depending on the determined net inspiratory volume flow. Depending on the control, the inspiratory display element is configured to display at least two indicators and thus the following two pieces of information in at least one way that a person can perceive visually:
an indicator for the magnitude (amount) of the determined net inspiratory volume flow and
an indicator for the direction of flow of the gas mixture through the inspiratory fluid guide unit.

In the simplest case, the indicator for the net inspiratory volume flow indicates whether or not a fluid with a volume flow above a specified lower threshold is flowing through the inspiratory fluid guide unit. It is also possible for this indicator to additionally display a continuous or stepped measure (indicator) of the magnitude (amount) of the volume flow.

The flow direction indicator shows whether fluid is flowing from the ventilator through the inspiratory fluid guide unit to the patient-side coupling unit or in the opposite direction. If there is no net inspiratory volume flow above a predetermined lower threshold, preferably a different flow direction indicator or no flow direction indicator at all is displayed.

Various errors and malfunctions can occur during artificial ventilation. These errors and malfunctions can put an artificially ventilated patient at risk. It is therefore important that a user recognizes these errors and malfunctions quickly. Examples of such errors and malfunctions that a user can recognize and, in many cases, eliminate due to the invention are the following:
A fluid connection between the ventilator and the patient-side coupling unit was not established by mistake.
A fluid guide unit is not connected to the correct connection element on the ventilator, but to a wrong one.

Because a fluid guide unit with a diameter that is too small is used, the volume flow actually achieved is smaller than the required volume flow.
A fluid guide unit or the patient-side coupling unit is kinked or blocked (clogged) or incorrectly closed, so that the actual volume flow is less than the required volume flow or even incorrectly no fluid flows at all.
A fluid guide unit is not connected to the ventilator in a fluid-tight manner, so that part of the gas mixture ejected during a ventilation stroke is released or escapes into the environment instead of being directed to the patient-side coupling unit.
A leak in a fluid guide unit or a clogged filter leads to a volume flow that is too large or too small, and the volume flow to the patient-side coupling unit is therefore too small.
The ventilator is not configured to eject (expel, supply) the required amount of gas mixture, but ejects too little or no gas mixture at all.
Due to an incorrect user setting (specification), an incorrect volume flow setting has been set on the ventilator.

The invention has the effect that the current internal status of a device is displayed in an ergonomic manner, namely a status of the ventilation system.

The respective volume flow through several fluid guide units of a ventilation system may be displayed on a central display unit. The invention can be combined with such a central display unit. However, this embodiment requires the central display unit to additionally indicate which output (displayed) value relates to which fluid guide unit. This information could be displayed textually and/or graphically. An output on a central output unit requires more space and in some cases more computing capacity than the output of information according to the invention. Especially in clinical practice, the space available for a central display unit is often limited. Furthermore, the use of a central output unit requires that a user understands which output value for a volume flow refers to which fluid guide unit.

In contrast, the invention eliminates the need to use a central display unit to output information about an inspiratory volume flow, thereby saving space. In addition, the invention eliminates the need for a user to mentally associate a representation of a fluid guide unit in a central display with the real (physical) fluid guide unit. According to the invention, the information about a volume flow through a fluid guide unit is displayed directly on this fluid guide unit. In this way, the invention reduces the risk of errors, in particular the risk of a user not finding information or assigning it incorrectly. In addition, the user can often grasp the information displayed according to the invention more quickly.

According to the invention, the inspiratory display element displays the two indicators in a visually perceptible manner. It is possible that in the event of an incorrect volume flow, an alarm is additionally output acoustically and/or tactilely (haptically), preferably by vibrations, and/or displayed on a spatially remote display unit. However, the feature according to the invention that the inspiratory display element visually outputs two indicators, eliminates the need to report errors and malfunctions exclusively by acoustic or haptic alarms and/or alarms on a display unit of a spatially remote receiver. In many cases, an audible or haptic alarm only indicates that a fault has occurred, but not where the fault has occurred. In addition, a user is often exposed to a lot of noise in everyday clinical practice. A remote receiver is often relatively far away from the location where the fault has occurred. The invention eliminates the need to inform a user of a fault in the ventilation system solely by means of an acoustic or haptic alarm. Instead, the invention makes it possible to warn the user visually and, optionally, also acoustically and/or haptically.

According to the invention, a display element indicates the magnitude and direction of a volume flow. It is not necessary to specify in advance when this magnitude or direction is correct and when it is incorrect, as such there is no need for an alarm to be generated and output.

According to the invention, the control unit determines the net inspiratory volume flow and causes this to be displayed visually. The control unit therefore does not, or at least not only, determine the total volume flow through the inspiratory fluid guide unit, but determines that proportion of the total volume flow which is generated by the ventilator's ventilation strokes. This reduces the risk that a volume flow generated in another way simulates sufficient ventilator activity, even though the ventilator does not perform any ventilation strokes or enough ventilation strokes.

According to the invention, the control unit determines the net inspiratory volume flow through the inspiratory fluid guide unit, i.e. the volume flow generated by the ventilator. This net inspiratory volume flow occurs outside the ventilator. Therefore, it is not a volume flow within the ventilator that is displayed, but a volume flow between the ventilator and the patient-side coupling unit. The invention thus makes it possible to detect not only the effect of a fault in the ventilator, but also the effect of a fault between the ventilator and the patient-side coupling unit.

In one embodiment, the ventilation system comprises an inspiratory volume flow sensor. This inspiratory volume flow sensor is capable of determining a measure of the volume flow through the inspiratory fluid guide unit. The control unit receives and processes a signal from the inspiratory volume flow sensor to determine the measure of net inspiratory volume flow. The received and processed signal includes information about the volume flow. It is possible that the volume flow sensor automatically processes raw measured values, for example smoothing them, and/or recognizes and excludes outliers. In particular, the control unit aggregates several signal values from the inspiratory volume flow sensor for different measurement times. The aggregation includes, in particular, averaging or weighted averaging over several measured values.

In one embodiment, the control unit determines the inspiratory phases and/or an oscillating component in the signal from the inspiratory volume flow sensor. At least when the ventilation system is working correctly, an ejected quantity of the gas mixture flows from the ventilator to the patient-side coupling unit in each inspiratory phase. At least in each inspiratory phase, the oscillating portion is generated by the ventilator. The control unit determines the frequency and/or amplitude of the oscillating component.

In one embodiment, the control unit determines (acquires/captures) a target frequency and/or a target amplitude of the ventilation strokes from a higher-level controller. The control unit uses a comparison with the target value(s) to determine whether the oscillating component actually originates from the ventilator. Optionally, the control unit also uses knowledge of the inspiratory phases for this purpose.

The configuration with the volume flow sensor makes it possible to determine the volume flow through the inspiratory fluid guide unit directly at the inspiratory fluid guide unit and not at a spatially distant measuring point. A measurement at a remote measuring point can lead to greater errors than a measurement directly at the inspiratory fluid guide unit.

The inspiratory fluid guide unit connects the patient-side coupling unit to the ventilator. In one embodiment, no ventilation circuit is established, i.e. the air exhaled by the patient is released into the environment. In particular, if no ventilation circuit is established, the inspiratory volume flow sensor signal is a good approximation of the net inspiratory volume flow to be determined. In one embodiment, the control device uses the volume flow measured by the inspiratory volume flow sensor as the net inspiratory volume flow. In another embodiment, the control device uses signals from further sensors, each of which measures a variable indicative of further volume flows.

According to the invention, the control unit determines a measure of the net inspiratory volume flow. According to the preferred embodiment just described, the control unit uses a signal from the inspiratory volume flow sensor for this purpose. In one embodiment, the control unit derives a value for the current net inspiratory volume flow from the signal, preferably from a signal value, and uses this to control the inspiratory display element. In another form of implementation, the control unit averages several signal values from the inspiratory volume flow sensor or aggregates several values in another way, e.g. as a median, and preferably derives an averaged net inspiratory volume flow. In many cases, this form of implementation means that the volume flow indicator displayed by the display element is less dependent on fluctuations in the net inspiratory volume flow.

According to the invention, the inspiratory display element is attached to the inspiratory fluid guide unit or to a further fluid guide unit, wherein the further fluid guide unit is in a fluid connection with the ventilator and with the inspiratory fluid guide unit. In one embodiment, this further fluid guide unit is an inspiratory connection piece (connector), this connection piece being configured in particular as a tube. The inspiratory connection piece is a part of the ventilator and is preferably attachable or fixed to the outside of a housing of the ventilator, particularly preferably permanently fixed. The inspiratory fluid guide unit can be connected to the inspiratory connection piece in a detachable and fluid-tight manner. For example, a tube of the inspiratory fluid guide unit is pushed over the tube. Of course, it is possible that due to a fault, the inspiratory fluid guide unit is not connected to the inspiratory connection piece at all or is not connected in a fluid-tight manner, or that a filter upstream or downstream of a fluid guide unit is blocked, which in many cases a user can notice thanks to the invention. When the inspiratory fluid guide unit is connected to the inspiratory connection piece, a fluid connection is established between the inspiratory fluid guide unit and the ventilator.

This configuration makes it possible to attach the inspiratory display element to the ventilator. It is then not part of the inspiratory fluid guide unit and therefore cannot act on the inspiratory fluid guide unit. If the inspiratory fluid guide unit is disconnected from the inspiratory connection piece, the inspiratory display element remains on the ventilator. Although the inspiratory display element is not part of the inspiratory fluid guide unit, it is located close to the inspiratory fluid guide unit. It is configured to indicate whether and, if so, in which direction a fluid is flowing through the inspiratory connection piece and thus through the inspiratory fluid guide unit.

In one embodiment, the ventilation system establishes a ventilation circuit. This embodiment is preferably used in particular when the patient is anesthetized, the air exhaled by the patient therefore comprises at least one anesthetic agent and this anesthetic agent should not enter the environment. An expiratory fluid guide unit of the ventilation system directs a gas mixture that leaves the patient-side coupling unit, usually the air exhaled by the patient, to the ventilator. An expiratory display element (expiratory indicator element) is attached to the expiratory fluid guide unit or to another fluid guide unit, this other fluid guide unit being in fluid connection with the ventilator and with the expiratory fluid guide unit.

According to this embodiment, the control unit is configured to determine a measure of the expiratory volume flow. This expiratory volume flow is the volume flow that flows from the patient-side coupling unit through the expiratory fluid guide unit to the ventilator. The control unit is configured to control the expiratory display element depending on the expiratory volume flow determined. The expiratory display element is capable of displaying two indicators, depending on the activation by the control unit and in a manner that can be visually perceived by a human, namely the following two indicators:
an indicator for the magnitude (the amount) of the expiratory volume flow determined and
an indicator for the flow direction of the gas mixture flowing through the expiratory fluid guide unit.

In a simple embodiment, the expiratory display element indicates whether or not a fluid with a volume flow above a predefined lower threshold is flowing through the expiratory fluid guide unit.

The configuration with two different display elements that are physically (spatially) spaced apart from each other makes it even easier for a user to detect and localize a fault. An error can occur in particular in connection between the ventilator on the one hand and the inspiratory fluid guide unit and/or with the expiratory fluid guide unit on the other.

A form of implementation was described above in which the inspiratory display element is attached to an inspiratory connection piece of the ventilator, whereby the inspiratory fluid guide unit can be connected to the inspiratory connection piece in a fluid-tight and detachable manner. A corresponding form of implementation is also possible for the expiratory display element. The expiratory fluid guide unit can be connected to an expiratory connection piece in a detachable and fluid-tight manner, with the expiratory connection piece preferably being configured as a tube which is attached to the outside of a housing of the ventilator. The advantages described above with regard to the inspiratory connection piece can also be achieved for the expiratory connection piece.

If a ventilation circuit is established, the ventilator feeds a gas mixture that flows through the expiratory fluid guide unit to the ventilator back into the inspiratory fluid guide unit. Preferably, the ventilator extracts carbon dioxide from this gas mixture beforehand. The volume flow through the inspiratory fluid guide unit therefore comprises a superposition of the net inspiratory volume flow generated by the ventilator and the volume flow through the expiratory fluid guide unit, minus the volume flow of the extracted carbon dioxide.

In a preferred embodiment, the ventilation system therefore comprises an inspiratory volume flow sensor and an expiratory volume flow sensor. The inspiratory volume flow sensor measures a variable indicative of the total volume flow through the inspiratory fluid guide unit, while the expiratory volume flow sensor measures a variable indicative of the volume flow through the expiratory fluid guide unit. The optional inspiratory volume flow sensor generally measures a variable indicative of the volume flow resulting from the superposition mentioned above, optionally with at least one additional volume flow being added. The control unit determines the net inspiratory volume flow depending on one signal each from the inspiratory volume flow sensor and the expiratory volume flow sensor. In the embodiment in which a ventilation circuit is established, the ventilation system comprises the inspiratory display element according to the invention and the preferred expiratory display element described above. The features that the ventilation system according to this embodiment comprises two spatially separated display elements and that the inspiratory display element is controlled depending on the net inspiratory volume flow make it easier for a user to localize a fault or an error or malfunction. In particular, it is easier for a user to distinguish a fault in the inspiratory fluid guide unit from a fault in the expiratory fluid guide unit.

According to the invention, the inspiratory display element displays an indicator for the net inspiratory volume flow through the inspiratory fluid guide unit. In the simplest case, this indicator shows whether a volume flow above a predetermined lower threshold flows through the inspiratory fluid guide unit or not. In another implementation this indicator also shows a continuous or graduated indicator of the magnitude of the volume flow. This measure of the magnitude of the volume flow depends on the net inspiratory volume flow, which the control unit determines as described above.

In one implementation, this indicator of the volume flow is the same for each patient and depends only on the net inspiratory volume flow. In another implementation, however, it is taken into account that the net inspiratory volume flow can vary greatly from patient to patient, even in error-free operation. In particular, the net inspiratory volume flow in a child is generally much smaller than in an adult. The net inspiratory volume flow can also depend on the condition of the patient's lungs and/or on an artificial ventilation procedure. The following form of implementation makes it easier for a user to detect an error on the inspiratory fluid guide unit despite these differences.

According to this form of implementation, the control unit is configured to capture (determine/record) a measure for a maximum target volume flow. This maximum target volume flow is specified (predetermined) and should be generated by the ventilator by ejecting the gas mixture in the ventilation strokes. In particular, the maximum target volume flow can be specified by a user or by a higher-level control system. For example, the maximum target volume flow is derived depending on the magnitude, age and/or weight of the patient. The maximum target volume flow can be specified as a time course, in particular as an oscillating curve, or as a maximum value or average value. This maximum target volume flow usually varies from patient to patient. Ideally, the net inspiratory volume flow through the inspiratory fluid guide unit lies within a predetermined tolerance range below this maximum target volume flow. The control unit is configured to calculate a quotient, with the determined (actual) net inspiratory volume flow in the numerator and the captured maximum target volume flow in the denominator of this quotient. The control unit is configured to control the inspiratory display element depending on the calculated quotient. The indicator for the magnitude of the volume flow depends on the calculated quotient. This configuration saves a user from having to compare and evaluate a display on the display element with a maximum volume flow to be achieved.

A further development of this embodiment can be used in particular if a ventilation circuit is established and an expiratory fluid guide unit leads from the patient-side coupling unit back to the ventilator. An expiratory volume flow sensor is configured to measure a variable indicative of the volume flow through the expiratory fluid guide unit. The volume flow through the expiratory fluid guide unit should also generally be within a tolerance band below the maximum target volume flow. As a further quotient, the control unit is configured to calculate the quotient from the determined volume flow through the expiratory fluid guide unit and the maximum target volume flow and to control the expiratory display element depending on this further quotient.

According to the invention, the control device is configured to control the inspiratory display element. The controlled inspiratory display element displays two indicators. One indicator is an indicator (measure) of the magnitude (amount) of the net inspiratory volume flow through the inspiratory fluid guide unit. Different implementations are possible as to how the inspiratory display element displays this indicator.

In a first form of implementation, the control unit is configured to cause the inspiratory display element to light up with a variable brightness. Preferably, the greater the magnitude of the net inspiratory volume flow to be displayed, the greater the brightness. Preferably, the non-illuminated inspiratory display element indicates that no fluid with a volume flow above a lower volume flow threshold is flowing through the inspiratory fluid guide unit. It is possible that the inspiratory display element has several possible brightness levels. It is also possible that the control unit is configured to continuously change the brightness by means of a corresponding control.

In one embodiment, the respective maximum brightness of each display element is fixed and cannot be changed. In another embodiment, the maximum brightness can be changed. A default value for the maximum brightness is specified. This default value is reached, for example, when the determined volume flow is equal to the maximum target volume flow described above, except for a tolerance. A user can change this default value. It is also possible that a brightness sensor measures the brightness in the environment of the ventilation system and the control unit changes the respective maximum brightness of a display element depending on the measured brightness in the environment, namely in such a way that the greater the ambient brightness, the greater the maximum brightness of the display element. The configuration with the variable maximum brightness makes it easier to adapt the display element to ambient conditions and reduces the risk of a fault being overlooked because the brightness of the display element is too low or, conversely, that the display element is perceived as too bright or visually obscures or outshines another indicator light.

In a second form of implementation, the control unit can cause the inspiratory display element to flash and/or flicker at a variable frequency. This frequency is an indicator of the magnitude of the net inspiratory volume flow. Preferably, the greater the net inspiratory volume flow, the greater the frequency.

The two forms of implementation just described can be combined with each other. It is also possible to use a color in addition or instead as an indicator for the magnitude of the net inspiratory volume flow, for example one of the three colors green, yellow, red (traffic light function). Preferably, green indicates a volume flow within a target tolerance band, yellow indicates a volume flow outside the target tolerance band but still within a wider tolerance band, and red indicates a volume flow outside the wider tolerance band.

In one embodiment, the ventilation system also comprises a gas mixer. This gas mixer is capable of generating a gas mixture from at least two supplied gas components. At least one gas component is or comprises oxygen. It is possible that at least one further gas component is or comprises an anesthetic. The gas mixer is at least temporarily in a fluid connection with the ventilator, and the generated gas mixture is conveyed to and into the ventilator. The gas components are each provided by a stationary or mobile supply connection.

In a further embodiment of this configuration, a gas mixer display element is attached to a fluid guide unit. The fluid guide unit is located at an outlet of the gas mixer. The control unit is configured to control this gas mixer display element. The activated gas mixer display element displays at least one indicator, namely an indicator for the magnitude of the volume flow through the outlet from the gas mixer.

In one embodiment, the ventilation system also comprises a bypass fluid guide unit and a pneumatic switch. The bypass fluid guide unit bypasses the ventilator and is in a fluid connection with the patient-side coupling unit. The switch is configured to direct a gas mixture either into the ventilator or into the bypass fluid guide unit. In one embodiment, one input of the switch is in a fluid connection with the gas mixer just mentioned. This embodiment increases the flexibility of the ventilation system. The ventilation system can optionally be used
for artificial ventilation, in which the patient is ventilated by the ventilator and optionally completely anesthetized, or
for ventilation in which the patient draws in and breathes in a gas mixture using their own respiratory activity.

This gas mixture comes from the gas mixer just mentioned, for example.

According to the embodiment just mentioned, a bypass display element (a bypass indicator element) is attached to the bypass fluid guide unit or to a further fluid guide unit that is in a fluid connection with the bypass fluid guide unit. The control unit is configured to control this bypass display element. The bypass display element is capable of displaying at least one indicator in a visually perceptible manner, namely an indicator for the magnitude of the volume flow through the bypass fluid guide unit. Optionally, the bypass display element can additionally display an indicator for the direction in which a fluid flows through the bypass fluid guide unit. In many cases, however, this indicator for the direction of flow is not required on the bypass fluid guide unit because often no fluid flows from the patient-side coupling unit into the bypass fluid guide unit. Optionally, a volume flow sensor measures the volume flow through the bypass fluid guide unit. In one embodiment, this sensor measures an indicator of the volume flow from the gas mixer just mentioned.

The embodiment with the bypass display element further facilitates a user to find a possible error, both when the patient is supplied by the ventilator and when the patient receives and inhales a gas mixture from the bypass fluid guide unit.

According to the invention, the control device controls the inspiratory display element, and the controlled inspiratory display element displays two indicators, namely an indicator for the magnitude (the amount) of the determined net inspiratory volume flow and an indicator for the flow direction of the gas mixture. In one embodiment, the control unit additionally captures a measure for a required net inspiratory volume flow. This measure comprises at least the information that a gas mixture should now flow out of the ventilator into the inspiratory fluid guide unit. In one embodiment, the control unit captures this required net inspiratory volume flow by automatically sending a request to a higher-level control of the ventilation system and processing a response. By means of an activation, the control unit causes the activated inspiratory display element to additionally activate at least one of the two indicators depending on the captured required net inspiratory volume flow. In particular, at least one indicator is highlighted if the determined measure for the actual volume flow deviates significantly, i.e. by more than a specified tolerance, from the captured required volume flow. This is particularly the case if the control unit has detected that a gas mixture should flow out of the ventilator into the inspiratory fluid guide unit, but the volume flow actually determined is below a predefined lower threshold.

This configuration makes it even easier for a user to quickly identify an error.

The invention is described below by means of an embodiment example. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view showing the ventilator, the gas mixer and the connection to the patient, with artificial ventilation supplementing the patient's own breathing activity;

FIG. 2 is a schematic view showing a modification of the configuration of FIG. 1, in which the patient is anesthetized, and a ventilation circuit is established;

FIG. 3 is a perspective view showing the display elements for the inspiratory line and the expiration line;

FIG. 4 is a flow chart showing how the control of the inspiratory display element is derived; and

FIG. 5 is a perspective view showing the display element for the bypass line.

### DESCRIPTION OF PREFERRED EMBODIMENTS

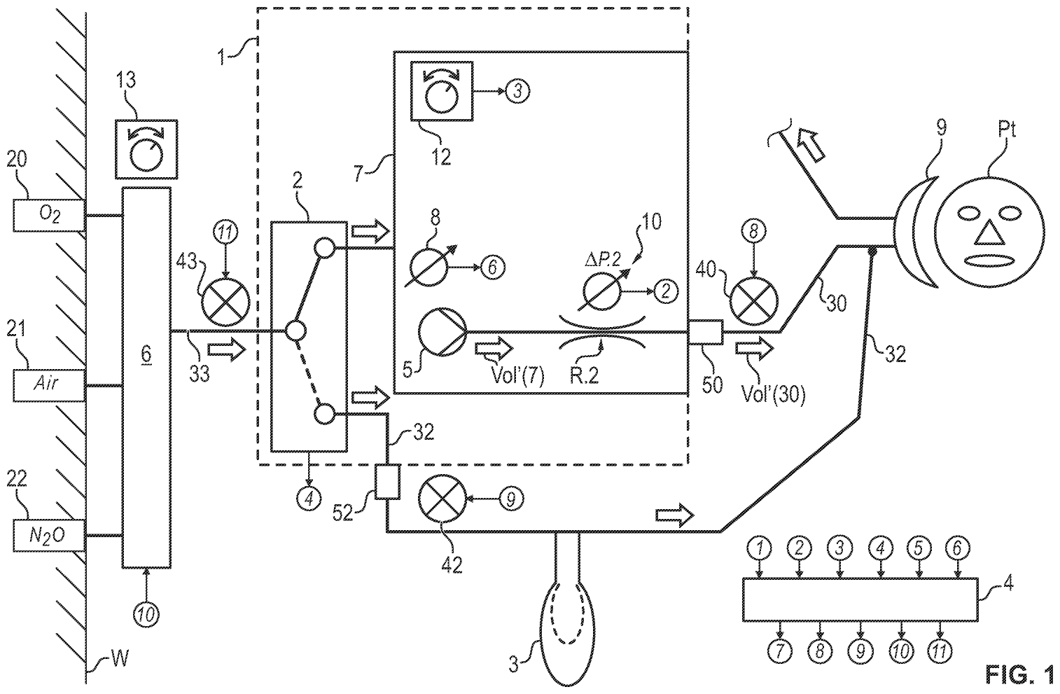

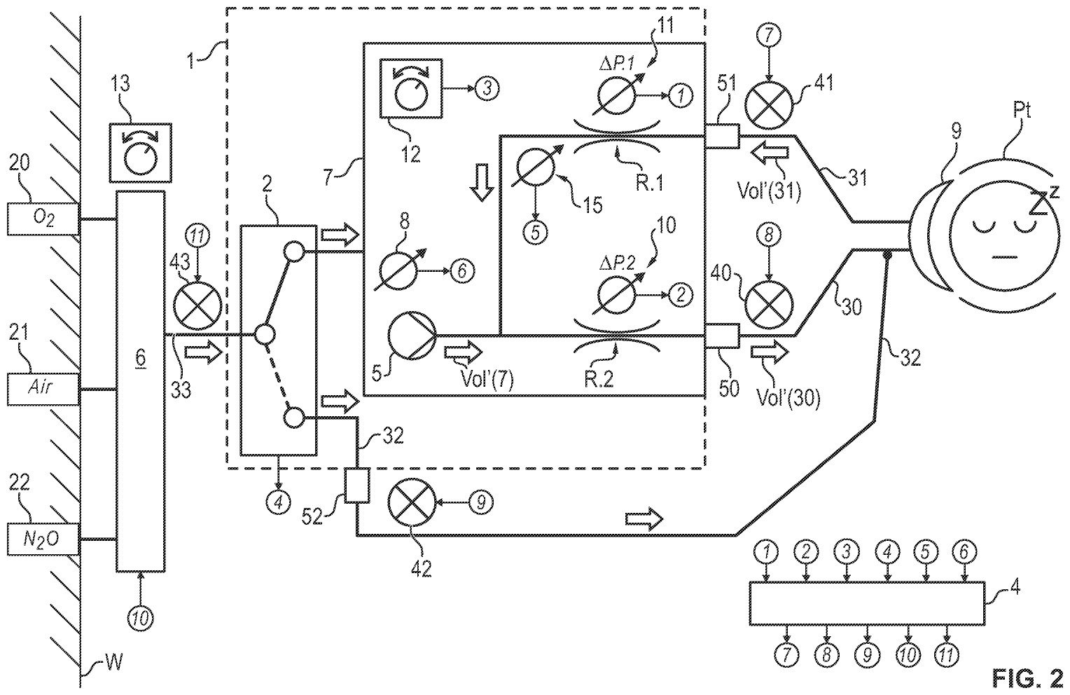

Referring to the drawings, FIG. 1 and FIG. 2 schematically show an application of the invention for the artificial ventilation of a patient Pt. Identical reference characters have the same meaning in FIG. 1 and FIG. 2. A patient-side coupling unit 9 is arranged at or on or in the body of the patient Pt. In the example shown, a breathing mask 9 is placed on the face of the patient Pt. In the example of FIG. 1, the patient Pt develops their own respiratory activity, which is carried out by the respiratory muscles of the patient Pt. This own respiratory activity can be spontaneous respiration and/or externally stimulated, for example in an electromagnetic field. A device that activates the respiratory muscles of a patient in such a field is described, for example, in WO 2019/154834 A1 (which is incorporated herein by reference). In the example in FIG. 2, however, the patient Pt is anesthetized or at least sedated.

An inspiratory gas mixture is supplied to the patient-side coupling unit 9 and thus to the patient Pt via an inspiratory line 30. The inspiratory gas mixture comprises oxygen and, in the application shown in FIG. 2, additionally at least one anesthetic agent (anesthetic). In the embodiment shown in FIG. 2, the expiratory gas mixture exhaled by the patient Pt is discharged from the patient-side coupling unit 9 via an expiratory line 31. The respective flow direction of the gas mixture is indicated by arrows in FIG. 1 and FIG. 2. Preferably, a Y-piece is arranged between the patient-side coupling unit 9 and the two lines 30 and 31. It is also possible that a two-lumen tube realizes both the inspiratory line 30 and the expiration line 31.

The inspiratory line 30 is attached to an inspiratory connection piece 50, the expiratory line 31 is attached to an expiratory connection piece 51. The two connection pieces 50, 51 are attached to the outside of a housing of a ventilator (respirator) 1 shown schematically. In the application shown in FIG. 1, the air exhaled by the patient Pt flows into the environment.

It is possible that a line 30, 31 is attached to an incorrect connection piece 51, 50 or incorrectly not attached to a connection piece at all.

The ventilator 1 comprises a drive unit 7, preferably with a pump and/or a blower 5. A signal-processing control unit 4, shown only schematically, controls the drive unit 7. The drive unit 7 causes the ventilator 1 to perform a sequence of ventilation strokes. In each ventilation stroke, the ventilator 1 ejects a quantity of the inspiratory gas mixture. The ejected quantity is conveyed through the inspiratory connection piece 50 into the inspiratory line 30 and through the inspiratory line 30 to the patient-side coupling unit 9. In the example in FIG. 2, the expiratory gas mixture flows from the patient-side coupling unit 9 through the expiratory line 31 and through the expiratory connection piece 51 back to the ventilator 1.

It is possible that the drive unit 7 does not work as desired. This can result in the inspiratory gas mixture not flowing through the inspiratory line 30, or not flowing through it with a sufficient volume flow, but for example at least part of it remains in the ventilator 1.

In the embodiment shown in FIG. 1, the ventilator 1 supports and supplements the patient's own respiratory activity Pt, and the expiratory gas mixture is conducted into the environment. In another embodiment, shown in FIG. 2, the ventilator 1 establishes a ventilation circuit in which the ventilator 1 removes carbon dioxide (CO₂) from the expiratory gas mixture and feeds the remaining gas mixture back into the inspiratory line 30. In one embodiment, an optional carbon dioxide sensor 15 measures a variable indicative of the concentration (proportion/fraction) of CO₂—of carbon dioxide—in the expiratory gas mixture flowing through the expiratory line 31.

A volume flow sensor 10 measures a variable indicative of the volume flow Vol′(30) through the inspiratory line 30. The volume flow is the volume per unit time of a fluid flowing through the inspiratory line 30. If a ventilation circuit is established, the volume flow sensor 10 measures a volume flow that comprises a superposition of the volume flow generated by the ventilation strokes of the drive unit 7 and the volume flow generated by the supply of the exhaled gas mixture (expiratory gas mixture), minus the volume flow of the carbon dioxide filtered out. A volume flow sensor 11 measures a variable indicative of the volume flow Vol′(31) through the expiratory line 31.

The following describes an exemplary implementation of the volume flow sensor 10. Other forms of implementation are also possible. The volume flow sensor 11 can be implemented in the same way.

A pneumatic resistor R.2 is arranged in the inspiratory line 30. The volume flow sensor 10 measures a variable indicative of the pressure difference ΔP.2 between the pressure upstream and the pressure downstream of the pneumatic resistor R.2. Accordingly, the volume flow sensor 11 measures a variable indicative of the volume flow Vol′(31) through the expiratory line 31, namely the pressure difference ΔP.1 between the pressure upstream and the pressure downstream of a pneumatic resistor R.1 in the expiratory line 31.

In a different embodiment, the volume flow Vol′(30) through the inspiratory line 30 is not measured by a volume flow sensor on the inspiratory line 30, but by a volume flow sensor in the ventilator 1. Alternatively, the volume flow Vol′(30) through the inspiratory line 30 is derived from a different signal, which is a measure of the volume flow generated by the drive unit 7. For example, a drive unit sensor 8 measures a signal that is indicative of the distance or angle of rotation that a fluid delivery unit of the drive unit 7, for example the pump 5, travels in a unit of time, or a measure of the volume flow at an output of the drive unit 7. It is also possible that both the volume flow sensor 11 on the inspiratory line 30 and the drive unit sensor 8 on the drive unit 7 each measure a variable indicative of the volume flow. In one embodiment, this achieves redundancy.

A gas mixer 6 generates the inspiratory gas mixture from various gas components. With the aid of an input unit 12 on the ventilator 1, a user can set a desired volume flow of the gas mixture which the gas mixer 6 provides and which flows out of the gas mixer 6 and to the ventilator 1. Using an optional input unit 13 on the gas mixer 6, the user can set the concentration of at least one gas component in the inspiratory gas mixture.

In the example shown, the gas mixer 6 is connected to a supply connection 20 for pure oxygen, a supply connection 21 for breathing air and a supply connection 22 for nitrous oxide (N₂O) or for another anesthetic. This number of supply connections and these noted gas components are to be understood as examples only. The three supply connections 20, 21, 22 are arranged stationary in a wall W in the example shown. It is also possible that the gas components come from containers, in particular from gas cylinders, which are preferably pressurized. Optionally, the ventilator 1 comprises a holder for each gas cylinder.

The gas components flow into the gas mixer 6 and are mixed there to form the inspiratory gas mixture. The gas mixer 6 can comprise an anesthetic evaporator or anesthetic vaporizer (not shown), which vaporizes the liquid anesthetic and mixes it with a carrier gas. The carrier gas is generated, for example, from gas components that provide the supply connections 20, 21, 22,

If the inspiratory gas mixture contains an anesthetic agent, the expiratory gas mixture also contains this anesthetic agent. In order to prevent anesthetic from entering the environment, a ventilation circuit is established in the embodiment shown in FIG. 2.

It is also possible that the ventilation strokes are not generated by the drive unit 7 of the ventilator 1, but by an optional manual ventilation bag 3, for example, which is operated by a person. This configuration is particularly useful if the ventilator 1 is currently inoperable. In particular in the embodiment shown in FIG. 1, it is also possible that the patient Pt is not sedated and their own respiratory activity is sufficient to supply the patient with sufficient breathing air, optionally in conjunction with artificial ventilation generated by the manual ventilation bag 3. In this case, the supply connections 20, 21, 22 are used, but not the drive unit 7 of the ventilator 1.

A bypass line 32 is connected to a bypass connection piece 52 on the ventilator 1, bypasses the drive unit 7 and the inspiratory line 30 and opens into the inspiratory line 30 (FIG. 1) or into an Y piece connected with the patient-side coupling unit 9 (FIG. 2). In one embodiment, the bypass connection piece 52 functions as a common gas outlet port. Instead of the bypass line 32, another line can also be connected to the bypass connection piece 52. In one embodiment, a volume flow sensor (not shown) is also arranged in the bypass line 32.

The ventilator 1 has a pneumatic switch 2 that can be actuated by a user. Depending on the position of this switch 2, the inspiratory gas mixture generated by the gas mixer 6 is directed either to the drive unit 7 or to the bypass connection piece 52 and from there on to the bypass line 42. In the position of switch 2 shown in FIG. 1 and FIG. 2, the gas mixture is directed to drive unit 7.

A supply fluid guide unit 33 connects the gas mixer 6 to the switch 2. A gas mixture generated by the gas mixer 6 flows through the supply fluid guide unit 33 to the switch 2.

The control unit 4 receives a signal from each of the volume flow sensors 10 and 11, from the drive unit sensor 8, from the CO₂ sensor 15, from the input units 12 and 13 and from the switch 2, processes the signals received and, depending on the result of the processing, controls the gas mixer 6, among other things. One aim of this control is to ensure that the inspiratory gas mixture provided by the gas mixer 6 has the composition specified by the user and that the specified volume flow is achieved. The control unit 4 is preferably located inside the ventilator 1.

In the embodiment there is arranged
an inspiratory display element 40 on the inspiratory connection piece 50 for the inspiratory line 30,
an expiratory display element 41 on the expiratory connection piece 51 for the expiratory line 31,
on the bypass connection piece 52 for the bypass line 32, a bypass display element 42 and
a display element 43 on the supply fluid guide unit 33.

Each display element 40, 41, 42, 43 is capable of indicating in at least one visually perceptible manner whether a fluid with a volume flow above a predetermined threshold is flowing through the connecting piece 50, 51, 52 to which the display element 40, 41, 42 is attached or with which the display element 40, 41, 42 is associated, as well as the direction of flow of this fluid, i.e. whether the fluid is flowing out of the ventilator 1 through the connecting piece 50, 51, 52 to the outside or, conversely, from the outside through the connecting piece 50, 51, 52 into the ventilator 1. Each display element 40, 41, 42 has at least one of the two possible states illuminated or not illuminated. The same applies to the display element 43.

Optionally, at least one display element 40, 41, 42, 43 is additionally capable of displaying an indicator for the magnitude (the amount) of the volume flow through the associated connection piece 50, 51, 52, 53. Preferably, the brightness of an display element 40, 41, 42, 43 depends on the volume flow, and the greater the volume flow, the greater the brightness. In an alternative embodiment, a display element 40, 41, 42, 43 flashes or flickers at a frequency that can be perceived by a human being, wherein preferably the frequency of the flashing depends on the volume flow and is particularly preferably greater the greater the volume flow. These two embodiments can be combined with each other.

In one form of implementation, the determined current volume flow through a line 30, 31, 32, 33 is used for the display described below. As a rule, the respective volume flow through a line 30, 31, 32, 33 varies. In an alternative form of implementation, the control unit 4 calculates a time-averaged volume flow, for example averaged over the duration of n ventilation strokes, where n>=1 is a predefined number, or as a median over the last n values. It is also possible to numerically integrate the volume flow over a predetermined period of time or over the duration of n ventilation strokes in order to form the average. Preferably, each display element 40, 41, 42, 43 represents an indicator for the averaged volume flow.

In one embodiment, the control unit 4 controls the display elements 40, 41, 42, 43 exclusively depending on the determined measure for the actual volume flow through the respective fluid guide unit, optionally also depending on a subsequent maximum target volume flow. In another embodiment, the control unit 4 captures a required flow direction through the respective fluid guide unit. For example, the control unit 4 automatically queries a higher-level control or regulation of the ventilation system, whereby this higher-level control or regulation has, for example, detected a setting from a user. If the measured flow direction deviates from the required flow direction, in particular if no fluid flows at all above a specified minimum volume flow threshold, the respective display element 40, 41, 42, 43 is highlighted.

The control unit 4 controls the display elements 40, 41, 42, 43 depending on signals from the volume flow sensors 10 and 11 and from other signals, which is described below. The control unit 4 is configured to set and change the state of each display element 40, 41, 42, 43 independently of the respective state of each other display element.

In one embodiment, each display element 40, 41, 42, 43 comprises at least one LED or other suitable light source. A pulsed electrical voltage is applied to each display element 40, 41, 42, 43. The control unit 4 preferably changes the respective brightness of each display element 40, 41, 42, 43 by means of pulse width modulation (PWM). Here, the length of the electrical pulses and/or the pause between two pulses is set and changed as required. Of course, the control unit 4 can also switch off a display element 40, 41, 42, 43.

In one embodiment, the ventilation system implements a ventilation circuit, see FIG. 2. According to the invention, the control unit 4 determines a net inspiratory volume flow Vol′ᵢₙₛₚ(50), i.e. the volume flow that the drive unit 7 achieves with the ventilation strokes. The total volume flow Vol′(30) through the inspiratory line 30 comprises a superposition of this net inspiratory volume flow Vol′ᵢₙₛₚ(50) and the volume flow that results from the expiratory gas mixture exhaled by the patient Pt being returned to the inspiratory line 30 after carbon dioxide has been filtered out. For example, the control unit 4 uses signals from the two volume flow sensors 10 and 11, from the drive unit sensor 8 and from the optional CO₂ sensor 15 or from a sensor not shown, which measures a variable indicative of the amount of carbon dioxide filtered out, to determine the net inspiratory volume flow Vol′ᵢₙₛₚ(50). The following exemplary description relates to an application in which a ventilation circuit is established.

In another embodiment, breathing air flows from the gas mixer 6 through the bypass line 32 directly to the patient-side coupling unit 9. In this embodiment, the air exhaled by the patient Pt is preferably released into the environment, see FIG. 1.

In one embodiment, an additional gas flows through at least one line 30, 31, 32, 33, which does not flow to or from the patient Pt, but is used, for example, for cleaning the line 30, 31, 32, 33 or for measuring a gas component in a gas mixture. The control unit 4 computationally compensates for the influence of this volume flow on the inspiratory volume flow or the expiratory volume flow.

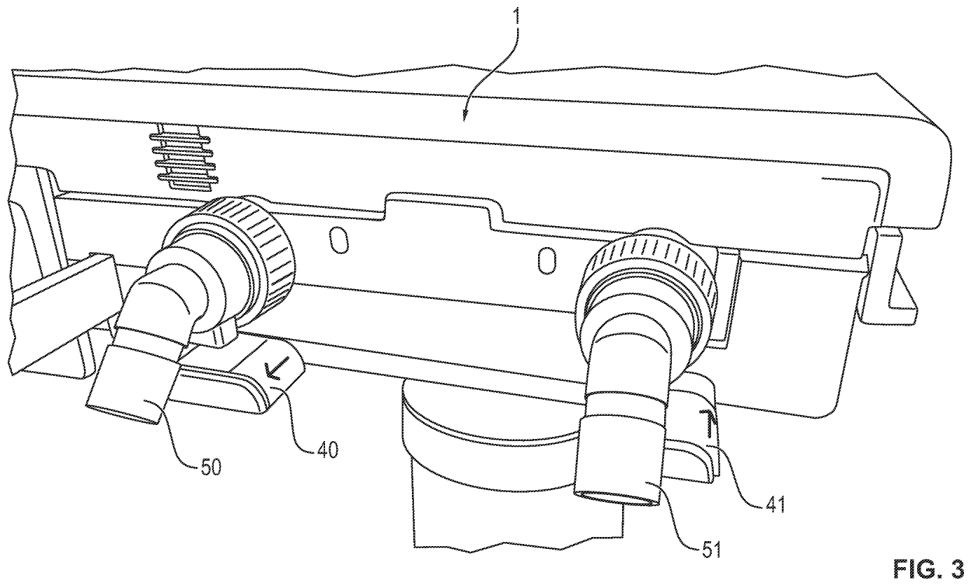

FIG. 3 shows an example of the display element 40 on the inspiratory connection piece 50 and the display element 41 on the expiratory connection piece 51. A hose can be attached to and removed from each of these connection pieces 50 and 51. Each display element 40, 41 is illuminated or not illuminated depending on the control by the control unit 4. An arrow on the display element 40, 41 indicates the current flow direction of fluid through the respective connection piece 50, 51. Optionally, the greater the volume flow through the connection piece 50, 51, the brighter the display element 40, 41.

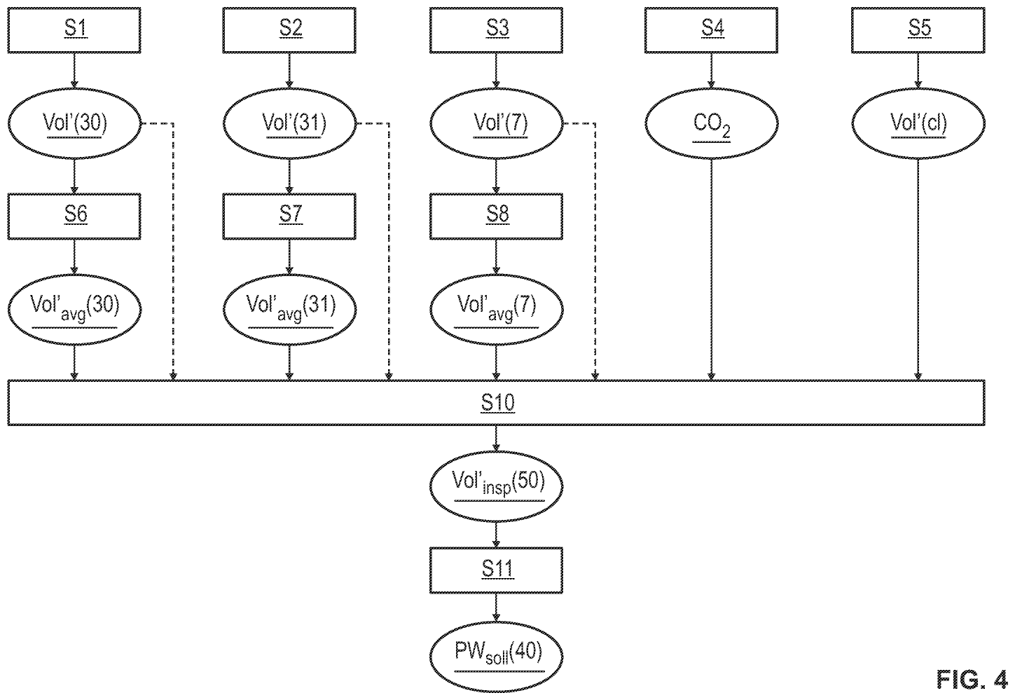

FIG. 4 uses a flow diagram as an example to illustrate how a default value for the brightness is automatically derived, whereby the display element 40 on the inspiratory connection piece 50 uses the derived brightness to visualize that portion of the volume flow through the inspiratory connection piece 50 that is generated by the drive unit 7, i.e. the net inspiratory volume flow Vol′ᵢₙₛₚ(50). This means:

| | |
|---|---|
| S1 | Step: The volume flow sensor 10 measures the volume flow Vol′(30) |
| | through the inspiratory line 30. |
| S2 | Step: The volume flow sensor 11 measures the volume flow Vol′(31) |
| | through the expiratory line 31. |
| S3 | Step: The drive unit sensor 8 measures the volume flow Vol′(7) |
| | generated by the drive unit 7. |
| S4 | Step: The CO₂ sensor 15 measures a variable indicative |
| | of the concentration CO₂ of carbon dioxide in the |
| | expiratory gas mixture flowing through the expiratory line 31. |
| S5 | Step: A measure for the averaged purging (flushing) and/or measuring |
| | volume flow Vol′(cl) is determined. |
| S6 | Step: The control unit 4 calculates a time-averaged volume flow |
| | Vol′_{avg}(30) through the inspiratory line 30. |
| S7 | Step: The control unit 4 calculates a time-averaged volume flow |
| | Vol′_{avg}(31) through the expiratory line 31. |
| S8 | Step: The control unit 4 calculates a time-averaged volume flow |
| | Vol′_{avg}(7) generated by the drive unit 7. |
| S10 | Step: The control unit 4 calculates the averaged net inspiratory volume |
| | flow Vol′ᵢₙₛₚ(50) through the inspiratory connection piece 50, which |
| | is generated by the drive unit 7 and visualized by the display |
| | element 40. |
| S11 | Step: The control unit 4 calculates a target pulse width PWₛₒₗₗ(40) |
| | for the display element 50. |

Note: If no error occurs, the averaged net inspiratory volume flow Vol′ᵢₙₛₚ(50) is ideally equal to the averaged volume flow Vol′_{avg}(7).

Steps S6, S7 and S8 are optional steps. In FIG. 4, dashed arrows indicate the sequence if these steps are omitted.

Preferably, the control unit 4 determines the net inspiratory volume flow Vol′ᵢₙₛₚ(50) in step S10 based on the volume flows Vol′(30), Vol′(31) and Vol′(cl). It is preferably assumed that all carbon dioxide is filtered out of the expiratory gas mixture and that the expiratory gas mixture without carbon dioxide is completely fed back into the inspiratory line 30. Based on this, the following applies:

$${Vol}^{′} ( 30 ) = {Vol}_{insp}^{′} ( 50 ) + ( 1 - {CO}_{2} ) * {Vol}^{′} ( 31 ) + {Vol}^{′} ( cl ) .$$

This results in the calculation rule

$${Vol}_{insp}^{′} ( 50 ) = {Vol}^{′} ( 30 ) - ( 1 - {CO}_{2} ) * {Vol}^{′} ( 31 ) - {Vol}^{′} ( cl ) .$$

The volume flow Vol′_{avg}(7) is used for a plausibility check, which the control unit 4 performs automatically. Ideally, the following applies:

$${Vol}_{avg}^{′} ( 7 ) = {Vol}^{′} ( 30 ) - ( 1 - {CO}_{2} ) * {Vol}^{′} ( 31 ) - {Vol}^{′} ( cl ) .$$

If there is a significant deviation between the left and right sides of this equation, there is an error, for example a measurement error or a sensor failure.

One embodiment takes into account the fact that the maximum target volume flow that the drive unit 7 should achieve with the ventilation strokes can vary from patient to patient. In particular, the maximum target volume flow for a child is significantly lower than for an adult. This target volume flow is preferably taken into account in order to prevent the following undesirable effect: If the target volume flow were not taken into account, a display element 40, 41, 42, 43 would only light up weakly and/or with a low frequency when the maximum target volume flow is relatively low, even if the time-averaged actual net inspiratory volume flow Vol′ᵢₙₛₚ(50) reaches the maximum target volume flow. The state of the display element 40, 41, 42, 43 would then be relatively difficult to determine, particularly in the case of significant ambient lighting. One could wrongly come to the conclusion that no or too little fluid is flowing at all.

Preferably, the control unit 4 captures a measure for the maximum target volume flow, for example from a higher-level control or regulation system that specifies a time course of the target volume flow through the inspiratory line 30, or on the basis of a setting by a user. In a simple embodiment, the control unit 4 acquires and/or determines the weight and/or age and/or date of birth of the patient Pt, for example from data about the patient Pt entered by a user or from a database containing patient data. This data includes, in particular, the age, weight and height of the patient Pt. The control unit 4 derives a rough estimate of the maximum target volume flow from the data collected about the patient Pt using a predefined table. The brightness and/or the frequency with which the two display elements 40 and 41 light up and/or flicker depends on the quotient of the determined actual net inspiratory volume flow Vol′ᵢₙₛₚ(50) and the captured maximum target volume flow during ventilation of a specific patient Pt. In one embodiment, the brightness and/or frequency of the display element 41 on the expiratory connection piece 51 depends on the quotient between the time-averaged volume flow Vol′_{avg}(31) through the expiratory line 31 and the maximum target volume flow.

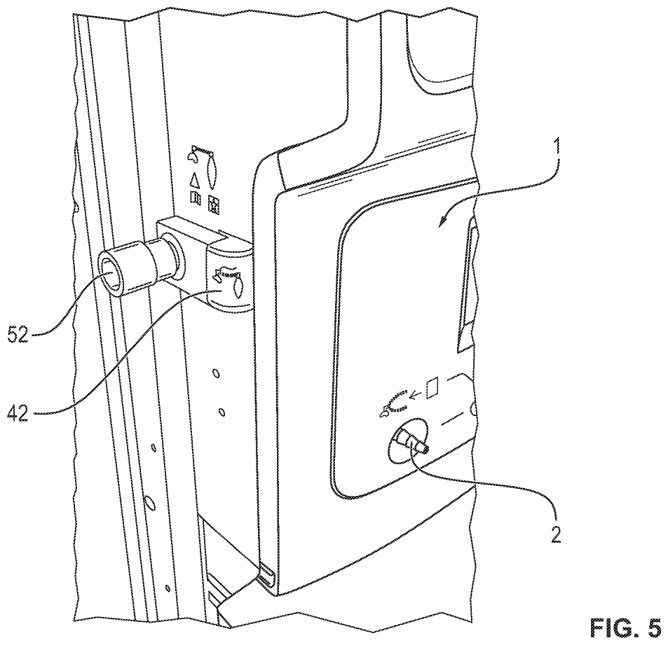

FIG. 5 shows the bypass display element 42 at the bypass connection piece 52. In one embodiment, a further volume flow sensor (not shown) measures the volume flow in or through the bypass line 32, and the brightness of the bypass display element 42 depends on the measured volume flow. In another embodiment, the brightness of the bypass display element 42 depends on the desired volume flow, which is predetermined with the aid of the input unit 12. It is also possible that the bypass display element 42 has only two possible states, namely illuminated and non-illuminated. In one embodiment, the display element 42 only lights up when the switch 2 is switched on so that it directs a gas from the gas mixer 6 into the bypass line 32. In another embodiment, the current state of the bypass display element 42 depends only on the measured or predetermined volume flow, but not on the position of the switch 2.

While specific embodiments of the invention have been shown and described in detail to illustrate the application of the principles of the invention, it will be understood that the invention may be embodied otherwise without departing from such principles.

List of reference characters

| | |
|---|---|
| 1 | Ventilator, comprises the drive unit 7 with the pump 5, the input unit |
| | 12, the switch 2, the volume flow sensors 10 and 11 and the display |
| | elements 40, 41 and 42 |
| 2 | Pneumatic switch to direct the gas mixture either to the ventilator 1 or |
| | directly through the bypass line 32 to the patient-side coupling unit 9 |
| 3 | Optional manual ventilation bag, pneumatically connected to the bypass |
| | line 32 |
| 4 | Signal-processing control unit, receives signals from the switch 2, from |
| | the volume flow sensors 10 and 11, 11, the sensors 8 and 15, the input |
| | units 12 and 13 and the switch 2, controls the display elements 40, 41 |
| | and 42 and the gas mixer 6 |
| 5 | Fluid delivery unit in the form of a pump, belongs to the drive unit 7 |
| 6 | Gas mixer, generates a gas mixture of pure oxygen, breathing air and |
| | nitrous oxide (N₂O), is connected to the supply |
| | connections 20, 21, 22 and to switch 2 |
| 7 | Drive unit of the ventilator 1, comprises the pump 5 |
| 8 | Drive unit sensor that measures a variable indicative of the volume flow |
| | generated by the drive unit 7 |
| 9 | Patient-side coupling unit in the form of a breathing mask, positioned |
| | on the patient's face Pt |
| 10 | Inspiratory volume flow sensor, measures a pressure difference at the |
| | pneumatic resistance R.2 as a measure of the volume flow through the |
| | inspiratory line 30 |
| 11 | Expiratory volume flow sensor, measures a pressure difference at the |
| | pneumatic resistance R.1 as a measure of the volume flow through the |
| | expiratory line 31 |
| 12 | Input unit on the ventilator 1, with which a user specifies a desired |
| | volume flow of the gas mixture to be provided by the gas mixer 6 |
| 13 | Input unit on the gas mixer 6, with which a user specifies a desired |
| | mixing ratio in the gas mixture which the gas mixer 6 is to produce |
| 15 | Carbon dioxide sensor, measures a variable indicative of the |
| | concentration (proportion) of carbon dioxide in the expiratory gas |
| | mixture flowing through the expiratory line 31 |
| 20 | Supply connection for pure oxygen in the wall W |
| 21 | Supply connection for breathing air in the wall W |
| 22 | Supply connection for nitrous oxide (N₂O) in the wall W |
| 30 | Inspiratory line, conducts a gas mixture from the ventilator 1 to the |
| | patient-side coupling unit 9 |
| 31 | Expiratory line, conducts exhaled air from the patient-side coupling unit |
| | 9 to the ventilator 1 |
| 32 | Bypass line, directs a gas mixture from the switch 2 into the inspiratory |
| | line 30 or to the patient-side coupling unit 9 |
| 33 | Supply fluid guide unit, feeds a gas mixture from the gas mixer 6 to the |
| | switch 2 |
| 40 | Display element on the inspiratory connection piece 50 for the |
| | inspiratory line 30, is operated with the set pulse width PWₛₒₗₗ(40) |
| 41 | Display element on the expiratory connection piece 51 for the |
| | expiratory line 31 |
| 42 | Display element on bypass connection piece 52 for bypass line 32 |
| 43 | Display element on the supply fluid guide unit 33 |
| 50 | Inspiratory connection piece, to which the inspiratory line 30 can be |
| | connected, connected to the display element 40 |
| 51 | Expiratory connection piece, to which the expiratory line 31 can be |
| | connected, connected to the display element 41 |
| 52 | Bypass connection piece, to which the bypass line 31 can be connected, |
| | connected to the display element 42 |
| CO₂ | Proportion of carbon dioxide in the expiratory gas mixture flowing |
| | through expiratory line 31 measured by sensor 15 |
| ΔP.1 | Pressure difference at pneumatic resistor R.1, measured by sensor 11 |
| ΔP.2 | Pressure difference at pneumatic resistor R.2, measured by sensor 10 |
| Pt | Patient who is artificially ventilated and anesthetized in one |
| | configuration wears the patient-side coupling unit 9 on the patient's |
| | face |
| PWₛₒₗₗ(40) | Set pulse width calculated by control unit 4, determines the brightness |
| | of display element 40 |
| R.1 | Pneumatic resistance in the expiratory line 31 |
| R.2 | Pneumatic resistance in the inspiratory line 30 |
| S1 | Step: The volume flow sensor 10 measures the volume flow Vol′(30) |
| | through the inspiratory line 30 |
| S2 | Step: The volume flow sensor 11 measures the volume flow Vol′(31) |
| | through the expiratory line 31 |
| S3 | Step: The volume flow sensor 8 measures the volume flow Vol′(7) |
| | generated by the drive unit 7 |
| S4 | Step: The CO₂ sensor 15 measures a variable indicative of the |
| | concentration of CO₂ carbon dioxide in the expiratory gas mixture |
| | flowing through the expiratory line 31 |
| S5 | Step: A measure for the averaged purging and/or measurement volume |
| | flow Vol′(cl) is determined |
| S6 | Optional step: The control unit 4 calculates a time-averaged volume |
| | flow Vol′_{avg}(30) through the inspiratory line 30 |
| S7 | Optional step: The control unit 4 calculates a time-averaged volume |
| | flow Vol′_{avg}(31) through the expiratory line 31 |
| S8 | Optional step: The control unit 4 calculates a time-averaged volume |
| | flow Vol′_{avg}(7) generated by the drive unit 7 |
| S10 | Step: The control unit 4 calculates the net inspiratory volume flow |
| | Vol′ᵢₙₛₚ(50), optionally the averaged volume flow, through the |
| | inspiratory connection piece 50, which is generated by the drive unit 7 |
| | and visualized by the display element 40 |
| S11 | Step: The control unit 4 calculates a target pulse width PWₛₒₗₗ(40) for |
| | the display element 50 depending on the net inspiratory volume flow |
| | Vol′ᵢₙₛₚ(50) |
| Vol′(30) | Volume flow through the inspiratory line 30, measured by the volume |
| | flow sensor 10, comprises a superposition of the net inspiratory volume |
| | flow Vol′ᵢₙₛₚ(50) and the expiratory volume flow Vol′(31) and |
| | optionally the purge and/or measurement volume flow Vol′(cl) |
| Vol′(31) | Volume flow through the expiratory line 31, measured by the volume |
| | flow sensor 11 |
| Vol′(7) | Volume flow generated by the drive unit 7 is measured by the drive unit |
| | sensor 8 |
| Vol′(cl) | Volume flow that occurs when purging (flushing) the inspiratory line |
| | 30 and optionally when tapping a gas sample from the inspiratory line |
| | 30 |
| Vol′_{avg}(30) | Time-averaged volume flow through the inspiratory line 30, is a |
| | superposition of the averaged net inspiratory volume flow Vol′ᵢₙₛₚ(50) |
| | and the averaged expiratory volume flow Vol′_{avg}(31) |
| Vol′_{avg}(31) | Time-averaged expiratory volume flow through the expiratory line 31 |
| Vol′_{avg}(7) | Time-averaged volume flow generated by the drive unit 7 |
| Vol′ᵢₙₛₚ(50) | Time-averaged net inspiratory volume flow through the inspiratory |
| | connection piece 50, determined by the control unit 4 |
| W | Wall in which the supply connections 20, 21, 22 are embedded |

What is claimed is:

1. A ventilation system for the artificial ventilation of a patient, the ventilation system comprising:
a ventilator configured to eject a gas mixture comprising oxygen;
an inspiratory fluid guide unit configured to be at least temporarily connected to the patient or to be connectable to the patient;
a patient-side coupling unit, the inspiratory fluid guide unit being configured to guide the gas mixture ejected by the ventilator to the patient-side coupling unit;
an inspiratory display element connected to the inspiratory fluid guide unit or to another fluid guide unit which is at least temporarily in fluid connection with the ventilator and with the inspiratory fluid guide unit; and
a signal-processing control unit configured:
to determine a measure for the net inspiratory volume flow, wherein the net inspiratory volume flow is the volume flow through the inspiratory fluid guide unit which volume flow the ventilator generates by ejecting the gas mixture and constitutes a portion of the total volume flow is through the inspiratory fluid guide unit; and
to control the inspiratory display element depending on the determined net inspiratory volume flow,
wherein the inspiratory display element is configured to visually perceptibly display, depending on the control, an indicator of a magnitude of the determined net inspiratory volume flow and an indicator of a direction of flow of the gas mixture through the inspiratory fluid guide unit.

2. A ventilation system according to claim 1, further comprising an inspiratory volume flow sensor that is configured to measure a variable indicative of the volume flow through the inspiratory fluid guide unit,
wherein the control device is configured to determine the measure of the net inspiratory volume flow using a signal from the inspiratory volume flow sensor.

3. A ventilation system according to claim 2, wherein the control unit is configured:
to determine an oscillating component in the signal of the inspiratory volume flow sensor;
to determine the frequency and/or the amplitude of the oscillating component, and
to use the determined frequency and/or amplitude for determining the net inspiratory volume flow.

4. A ventilation system according to claim 1,
wherein the ventilator further comprises an inspiratory connection piece,
wherein the inspiratory fluid guide unit is configured to be detachably and fluid tightly connected to the inspiratory connection piece,
wherein the inspiratory connection piece is configured to establish a fluid connection between the inspiratory fluid guide unit and the ventilator with a connection of the inspiratory fluid guide unit with the inspiratory connection piece being established, and
wherein the inspiratory display element is attached to the inspiratory connection piece.

5. A ventilation system according to claim 1, further comprising:
an expiratory fluid guide unit configured to guide a gas mixture leaving from the patient-side coupling unit to the ventilator; and
an expiratory display element, which is spatially remote from the inspiratory display element,
wherein the expiratory display element is attached to the expiratory fluid guide unit or to another fluid guide unit, which is at least temporarily in fluid connection with the ventilator and with the expiratory fluid guide unit,
wherein the control unit is configured:
to determine a measure for the expiratory volume flow, wherein the expiratory volume flow is the volume flow which flows through the expiratory fluid guide unit to the ventilator; and
to control the expiratory display element as a function of the determined expiration volume flow, and
wherein the expiratory display element is configured to visually perceptibly display, depending on the control, an indicator of a magnitude of the determined expiratory volume flow and an indicator of the flow direction of the gas mixture flowing through the expiratory fluid guide unit.

6. A ventilation system according to claim 5,
wherein the ventilator further comprises an inspiratory connection piece,
wherein the inspiratory fluid guide unit is configured to be detachably and fluid tightly connected to the inspiratory connection piece,
wherein the inspiratory connection piece is configured to establish a fluid connection between the inspiratory fluid guide unit and the ventilator with a connection of the inspiratory fluid guide unit with the inspiratory connection piece being established,
wherein the inspiratory display element is attached to the inspiratory connection piece,
wherein the ventilator further comprises an expiratory connection piece which is spatially remote from the inspiratory connection piece,
wherein the expiratory fluid guide unit is configured to be detachably and fluid tightly connected to the expiratory connection piece,
wherein the expiratory connection piece is configured to establish a fluid connection between the expiratory fluid guide unit and the ventilator with a connection of the expiratory fluid guide unit with the expiratory connection piece being established, and
wherein the expiratory display element is attached to the expiratory connection piece.

7. A ventilation system according to claim 5, further comprising:
an inspiratory volume flow sensor configured to measure a variable indicative of the volume flow through the inspiratory fluid guide unit; and
an expiratory volume flow sensor configured to measure a variable indicative of the volume flow through the expiratory fluid guide unit,
wherein the ventilation system is configured to establish at least temporarily a ventilation circuit between the patient-side coupling unit and the ventilator,
wherein the ventilation circuit comprises circuit flow through the inspiratory fluid guide unit and through the expiratory fluid guide unit, and
wherein the control device is configured to determine the measure for the net inspiratory volume flow based on the measured volume flow through the inspiratory fluid guide unit and based on the measured volume flow through the expiratory fluid guide unit.

8. A ventilation system according to claim 1, wherein the control unit is configured:
to capture a measure for a predetermined maximum target volume flow which the ventilator is to generate by ejecting the gas mixture; and
to control the inspiratory display element depending on a quotient between the determined net inspiratory volume flow and the captured maximum target volume flow.

9. A ventilation system according to claim 1,
wherein the displayed indicator for the determined net inspiratory volume flow comprises a degree of brightness of the inspiratory display element and/or a frequency of a flashing or flickering of the inspiratory display element,
wherein the ventilation system is configured such that the brightness and/or the frequency of a flashing or flickering depends on the determined net inspiratory volume flow.

10. A ventilation system according to claim 1, further comprising:
a pneumatic switch;
a bypass fluid guide unit which connects an output of the pneumatic switch to the patient-side coupling unit, wherein the pneumatic switch is configured to direct a mixture selectively into the ventilator or into the bypass fluid guide unit;
a bypass display element attached to the bypass fluid guide unit or to another fluid guide unit, which is at least temporarily in fluid connection with the ventilator and with the bypass fluid guide unit,
wherein the control unit is configured:
to determine a measure of the bypass volume flow, that is a volume flow through the bypass fluid guide unit, and
to control the bypass display element depending on the determined bypass volume flow, and
wherein the bypass display element is configured to visually perceptibly display, depending on the control, an indicator of a magnitude of the determined bypass volume flow and/or both an indicator of a magnitude of the determined bypass volume flow and an indicator of a direction of flow of the gas mixture through the bypass fluid guide unit.

11. A ventilation system according to claim 1,
wherein the control unit is configured:
to capture a measure of a required net inspiratory volume flow through the inspiratory fluid guide unit; and
to control the inspiratory display element additionally depending on the captured required net inspiratory volume flow,
wherein the inspiratory display element is configured to display at least one of the two indicators as a function of the captured required net inspiratory volume flow.

12. A ventilation system according to claim 11, wherein the determined measure of a required net inspiratory volume flow through the inspiratory fluid guide unit is based on a request to a control of the ventilator.
